(19) United States
(12) Patent Application Publication
(10) Pub. No.: US 2005/0089502 A1
(43) Pub. Date: Apr. 28, 2005

(54) Effervescent delivery system

(76) Inventors: Todd Schansberg, Jordan, MN (US); Jack B. Stubbs, Waynesville, OH (US)

Correspondence Address: John F. Klos;Fulbright & Jaworski L.L.P., 2100 IDS Center, 80 South Eighth Street, Minneapolis, MN, 55402-2112, US

(21) Appl. No.: 10/925,279

(22) Filed: Aug. 23, 2004

Related U.S. Application Data
(60) Provisional application No. 60/497,394, filed on Aug. 21, 2003.

(51) Int. Cl. A61K 7/46; A61L 9/01

(52) U.S. Cl. 424/76.1; 512/1

(57) ABSTRACT

An apparatus and method for delivering vapor from an effervescent material. The subject invention includes a housing, and a pouch containing an effervescent material. The effervescent chemical composition includes an exothermic reactant system and a fragrant material absorbed into an absorbent. When water is added to the reactant system it generates heat and volatizes the fragrant material, releasing the fragrance. The container includes one or more holes for allowing water to reach the reactant system and also includes one or more ridges for dispensing heat such that the container can be handled while the reactant system is releasing heat.

### RELATED APPLICATIONS

This application claims priority to U.S. Provisional Application No. 60/497,394 filed on Aug. 21, 2003, the contents of which are hereby incorporated in its entirety.

### FIELD OF THE INVENTION

Generally, the invention relates to an effervescent delivery system. More specifically, the invention relates to an effervescent fragrance delivery system including a housing therefore.

### BACKGROUND

The addition of pleasant fragrances to rooms, bathrooms, cars, and other areas is well known. Flowers, eucalyptus leaves, oils, and incense have been widely used for this purpose. Furthermore, it is known to add various types of “bath salts” to water when bathing. These materials add a pleasant aroma to enhance the bathing experience. A popular term for using various scents and aromas to soothe the body, both physically and mentally, is “aroma therapy.”

Various devices have been developed over the years for releasing fragrances into the air. These devices are often used in homes, offices, or other enclosed areas in order to not only add a pleasing fragrance into the air, but also to mask unpleasant odors. Simple systems, such as scented candles, for example, release fragrance from the melted wax. Burning candles, however, can be hazardous if left unattended, and are often prohibited in some areas (such as many office buildings).

“Potpourri” mixtures are also common. Traditional potpourri comprises various mixtures of aromatic herbs, dried flowers, and spices blended with essential oils. The aroma from the potpourri mixtures are typically emitted in a very confined space and last only a short time. Fragrant oils can also be sprayed or dripped onto the potpourri to enhance the aroma.

Traditional potpourri may also be used with an external heat source and water to cause the scent to become airborne through the vaporization process of boiling the water. One disadvantage is the requirement of an external heat source. The heated water becomes very hot and if it were to be spilled, it could badly burn the user. In addition, if all of the water in the simmerer is vaporized, the potpourri material itself can catch on fire.

Various other prior art systems for delivering scents include those shown in U.S. Pat. Nos. 6,548,015, 5,993,854, 6,432,450, 4,252,664, 4,941,483, 5,593,635, 5,885,701, and 5,041,421. These patents show a wide variety of oils and other materials in delivery systems designed to exude a pleasing smell.

U.S. Pat. No. 6,548,015 teaches a method and system of dispensing a fragrant vapor utilizing an exothermic reactant system contained with a volatile fragrance in a container. Water is added to the reactant system and the exothermic reaction generates heat to dispense the fragrance. The container is made of steel and may become very hot to the touch.

U.S. Pat. No. 5,993,854 shows an exothermic and effervescent material that promotes the release of a volatile agent with a fragrant smell. Effervescent compositions generally combine carbonate salts such as sodium carbonate and/or sodium bicarbonate with acidic materials such as citric, tartaric, or fumaric acid in a way that carbon dioxide is released when the product is placed in water. These products must be packaged in ways that prevent unintended contact with water so that a premature reaction is avoided. Even contact with humidity in the air must be prevented during manufacture and storage as this could detrimentally effect the effervescent properties.

U.S. Pat. No. 5,041,421 describes a fragrant material that imparts a pleasant fragrance when set out in ambient temperature or when simmered in water. The material is comprised of vacuum granulated sodium chloride wherein fragrant oils have been dispersed. The most effective way of releasing the fragrance is by heating the material using an external heat source.

### SUMMARY OF THE INVENTION

The present invention is directed to an effervescent delivery system 10 capable of releasing vapor at a controlled rate to sustain the delivery of a product over a period of time. The effervescent delivery system includes a pouch, a housing, and an effervescent chemical composition disposed within the pouch. The effervescent chemical composition includes a reactant system, a fragrant material.

In one embodiment, the pouch is preferably made of a filter paper or similar material that is porous to water but does not allow the chemical composition to pass through and spill. Preferably the filter paper should also not be affected by the temperatures generated by the exothermic reaction and should not be degraded by water. The pouch acts to limit the amount of water that contacts the effervescent chemical composition.

In one embodiment, the housing is a container that contains and protects the pouch while providing thermal insulation. The housing is comprised of a plurality of walls which cooperate to define an internal space wherein the pouch is disposed. A support structure is coupled to an interior surface of the walls to support the pouch. The material for the housing has integrity at the elevated service temperature of 100° C. or more, a temperature drop of 50° C. or more across its thickness, and has impermeability to steam. The ability to maintain integrity above a certain temperature is desired because of the heat produced by the exothermic reaction.

In one embodiment, the housing 14 also includes a number of raised surfaces or spaced apart ridges located on the external surface of the walls. The raised surfaces/ridges 28 provide additional insulation to enable the housing to be comfortably handled during the time the exothermic reaction is taking place and the aromatic vapor is being released.

In one embodiment, the housing may also be reusable, the pouch being inserted by the user into the housing just prior to initiation of the exothermic reaction. In one embodiment, the housing 14 has an internal surface that is adapted to position the pouch a predetermined distance from the sides of the housing. The support structure receives the pouch and positions the pouch away from the wall.

In one embodiment, the housing may further include one or more openings for addition of the water necessary to start the exothermic reaction. The openings in the housing acts as both filling ports and as steam vents for releasing vapor. The size and number of openings on the housing are selected to permit sufficient exposure to water to initiate and maintain the exothermic reaction and the controlled release of the vapor generated by the reaction.

In one embodiment, the effervescent delivery system includes a fragrant material disposed within the pouch. The fragrant material is comprised of a fragrant liquid that is impregnated into an absorbent material. In this manner, the liquid fragrance can be incorporated into the pouch 12 as a dry solid. In other embodiments, the fragrant material 24 may already be a solid, thus not requiring an absorbent material. In either case, the absorbent material may be included so as to help disperse the reactant system by providing separation between the components of the reactant system. Dispersing the reactant system 22 in this manner helps to control the rate of the exothermic reaction.

### BRIEF DESCRIPTION OF THE DRAWINGS

For purposes of facilitating and understanding the subject matter sought to be protected, there is illustrated in the accompanying drawings an embodiment thereof. From an inspection of the drawings, when considered in connection with the following description, the subject matter sought to be protected, its construction and operation, and many of its advantages should be readily understood and appreciated.

FIG. 1 is a perspective view of one embodiment of a fragrance delivery system in accordance with the present invention.

FIG. 2 is an exploded view of the system of FIG. 1, disclosing the interior surface of a housing and a pouch.

FIG. 3 is a longitudinal cross-sectional view of the system of FIG. 1.

FIG. 4 is a lateral cross-sectional view of the system of FIG. 1

### DETAILED DESCRIPTION

The present invention is directed to an effervescent delivery system 10 capable of releasing vapor at a controlled rate to sustain the delivery of a product over a period of time. The delivery system 10 can be used, for example, to generate an aromatic vapor for sensory, therapeutic, and/or medical purposes. In one embodiment, the delivery system 10 is capable of generating and sustaining the vapor release for the duration of a typical shower, or about 7-10 minutes, by the initiation and continuation of an exothermic reaction that heats a fragrance and releases fragrant vapor (in the form of steam) into the air. Other embodiments can include the ingredients in amounts adjusted to sustain vapor release for 30 minutes or more.

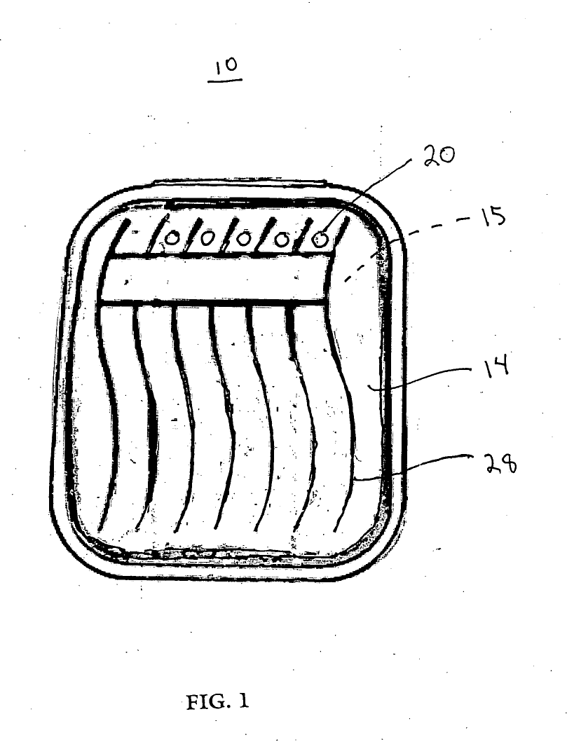

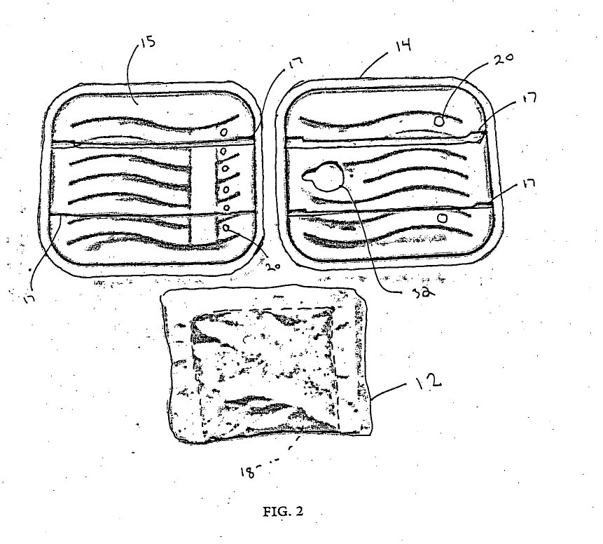

As illustrated in FIGS. 1 and 2, one embodiment of the effervescent delivery system 10 includes a pouch 12, a housing 14, and a chemical composition 18 disposed within the pouch 12. The chemical composition 18 includes a reactant system, a fragrant material, and, optionally, an absorbent material. The reactant system 22 provides the heat necessary to volatize the fragrant material 24. The absorbent material 26 is utilized to contain the fragrant material 24 and to absorb the water and to provide the water to the reactant system 22. The housing 14 is a container that contains the pouch 12. Specific features of the pouch 12, housing 14, and chemical composition 18 are further described below.

The pouch 12 is the lowest level of casing for the chemical composition 18. The material utilized to form the pouch 12 is preferably a filter paper or similar material that is porous to water but does not allow the chemical composition 18 to pass through and spill. Preferably the filter paper should also not be affected by the temperatures generated by the exothermic reaction and should not be degraded by water. In alternative embodiments, the filter paper may not be porous but may simply have an area that can allow water to enter the pouch 12 to reach the chemical composition 18.

One suitable filter paper is a heat sealable filter paper called Dexter® 11681 manufactured by the Dexter Corporation. This material is a lightweight blend of thermoplastic fibers that resists delamination when submersed in hot water. Other suitable materials for forming pouch 12 include woven materials, non-woven materials, apertured or perforated sheets and films, and solid materials such as metals, ceramics, glass, plastics, etc.

In one embodiment, the pouch 12 acts to limit the amount of water that contacts the chemical composition 18. The pouch 12 limits the maximum amount of water than can come into contact with the chemical composition 18 regulating the rate and duration of the exothermic reaction. In one embodiment, the pouch 12 absorbs water quickly, for example, when used in the shower, so that the chemical composition 18 can be quickly immersed and the exothermic reaction started.

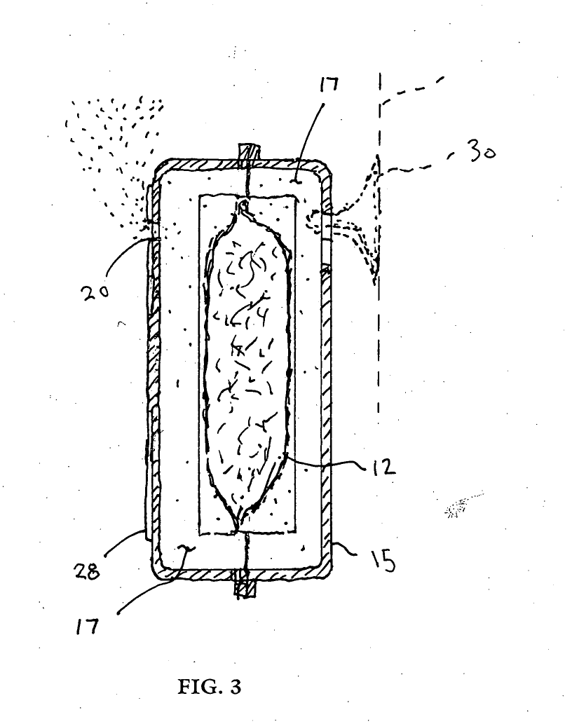

As shown in FIGS. 2-4, in one embodiment, the housing 14 is a container that contains and protects the pouch 12 while providing thermal insulation. The housing 14 is comprised of a plurality of walls 15 which cooperate to define an internal space wherein the pouch 12 is disposed.

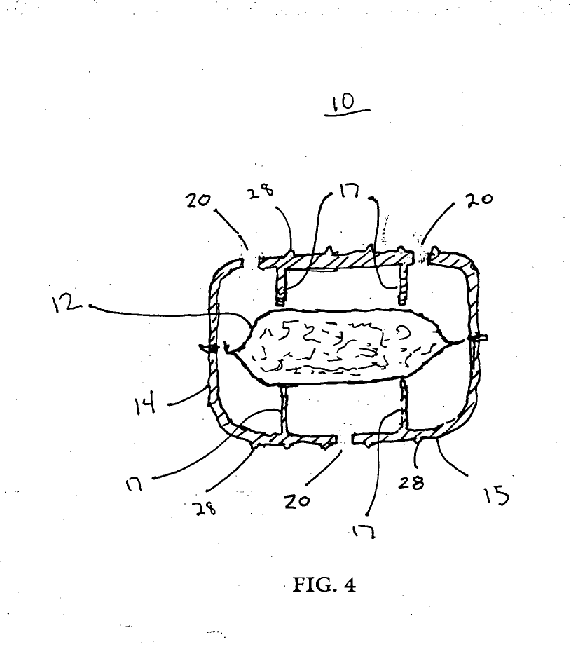

In one embodiment, a support structure 17 is coupled to an interior surface of the walls to support the pouch. The support structure 17 may be integral to the walls 15 and may be a formed component of a wall 15. Alternatively, the support structure may be connected to the walls 15. As shown in FIGS. 2 and 4, the support structure is a pair of C-shaped platforms extending from opposite walls 15. However, one skilled in the art would realize that the present invention would be amenable to a number of different support structures.

In one embodiment, the material for the housing 14 has integrity at the elevated service temperature of 100° C. or more, a temperature drop of 50° C. or more across its thickness, and has impermeability to steam. The ability to maintain integrity above a certain temperature is desired because of the heat produced by the exothermic reaction. The temperature drop across the thickness is preferred in order to allow for comfortable handling during the exothermic reaction. The housing 14 of the present invention preferably provides enough insulation for a temperature drop of at least about 30° C. across its thickness, more preferably at least about 40° C., and most preferably at least about 50° C. The housing 14 may have an outside surface temperature of less than 50° C. during the time in which the exothermic reaction is taking place. Finally, the impermeability to steam prevents the steam from escaping too quickly.

In one embodiment, the housing 14 is made of a polypropylene resin, but it may also be made of other materials, such as cross-linked polyethylene foam. Polypropylene resin is suitable for making the housing 14 because it is light, hard, and provides for a significant amount of thermal insulation. As will be apparent to those skilled in the art, the housing 14 and corresponding elements may be of any dimension suitable for containing and sustaining the vapor release during the desired period of time in the chosen environment.

In one embodiment, the housing 14 also includes a number of raised surfaces or spaced apart ridges 28 located on the external surface of the walls 15. The raised surfaces/ridges 28 provide additional insulation to enable the housing 14 to be comfortably handled during the time the exothermic reaction is taking place and the aromatic vapor is being released. The material of the housing 14 may provide for a temperature drop of up to 50° C. or more across its thickness, the ridges 28 provide an additional temperature drop.

In one embodiment, the housing 14 may also be reusable, the pouch 12 being inserted by the user into the housing 14 just prior to initiation of the exothermic reaction. In one embodiment, the housing 14 has an internal surface that is adapted to position the pouch a predetermined distance from the sides of the housing 14. The support structure 17 receives the pouch 12 and positions the pouch away from the walls 15.

As shown in FIGS. 1-4, in one embodiment, the housing 14 may further include one or more openings 20 for addition of the water necessary to start the exothermic reaction. The openings 20 in the housing 14 act as both filling ports and as steam vents for releasing vapor. The size and number of openings 20 on the housing 14 are selected to permit sufficient exposure to water to initiate and maintain the exothermic reaction and the controlled release of the vapor generated by the reaction.

Although the openings 20 are shown at the approximate center of the pouch, in other embodiments the opening may be positioned elsewhere. The openings 20 may also take on other forms, such as, but not limited to holes, perforations, cracks, apertures, or tears. These openings 20 may also be formed in desired shapes that are pleasing to the eye or that indicate the type of fragrance to be released. The housing 14 may contain a hole or other means for attaching the housing 14 to a surface. Such means may include a hole 32, a hook, a suction cup 30, or other temporary attachment means known to those in the art.

In one embodiment, the delivery system 10 includes an overwrap (not shown) comprised of a moisture and menthol/eucalyptus vapor barrier that covers each individual housing 14. The overwrap provides a vapor barrier around each housing 14 until it is removed prior to use. The overwrap barrier ensures that the pouch 12 is not exposed to moisture and therefore prevents the exothermic reaction from being prematurely activated. In addition, the overwrap prevents the selected fragrances from early dissipation, ensuring a quality product with long term storage capability.

In one embodiment, the effervescent delivery system includes a fragrant material disposed within the pouch. The fragrant material is comprised of a fragrant liquid that is impregnated into an absorbent material. In this manner, the liquid fragrance can be incorporated into the pouch 12 as a dry solid. In other embodiments, the fragrant material 24 may already be a solid, thus not requiring an absorbent material. In another embodiment, the fragrant material may be seperated from components of the reactant system and or absorbent material. In either case, the absorbent material may be included so as to help disperse the reactant system by providing separation between the components of the reactant system. Dispersing the reactant system 22 in this manner helps to control the rate of the exothermic reaction.

One example formulation of the present invention fragrance system 10 is as follows:

| Pouch percentage | | | Component % |
|---|---|---|---|
| 76% | Reactant | #2124 | |
| | | magnesium | 85-90% |
| | | iron | 2-5% |
| | | salt | 3-5% |
| | | plastic coating | <1% |
| 12% | Absorbent | Zeocal 250 | |
| | | calcium silicate | |
| 11% | Oil | A37021 | |
| | | Menthol | |
| | | Camphor | |
| | | Eucalyptus Oil | |
| | | Lavander Oil | |
| <1% | Filter paper | | |

As shown, the reactant system 22 includes appropriate amounts of magnesium, iron, and salt. The heat of reaction is a result of immersing the reactant system in an appropriate amount of water, which creates an electrolyte solution with the salt. The electrolyte solution allows the magnesium to act as an anode and the iron to act as a cathode in an exothermic chemical reaction. The salt is preferably present in a ratio of about 10% of the weight of the magnesium iron alloy to ensure that the reaction begins fairly quickly after wetting, thereby reassuring the consumer that the product is working. The addition of the salt to the reactant system 22 allows water alone to start the reaction. In order to meet Department of Transportation regulations, the combined magnesium iron alloy and salt mix is preferably kept under about 4 grams. This system is sometimes known as a super corroding metallic mixture.

Although magnesium iron alloy is the preferred reactant, any material which reacts exothermically with water may be used in the present invention. As will be apparent to those of skill in the art, if other exothermic reactants are used, the ratios and amounts of ingredients will vary depending on the rate and heat of reaction desired. Other metallic cathodes may include copper, cobalt, palladium, silver, gold, and platinum. Other anodes may include aluminum.

In one embodiment, the absorbent material utilized is Zeocal® 250, a precipitated amorphous calcium silicate powder. Other absorbents may utilized, such as vermiculite, silica or wood pulp. The absorbent is useful for containing the fragrant material if the fragrant material is a liquid. The fragrant material is absorbed into the absorbent's porous structure and then released when heated. The absorbent 26, as previously mentioned, serves to absorb the water that enables the exothermic reaction to occur. Water fill level is important, as too little water will cause the reaction to be over with too quickly, while with too much water, the reaction will not take place or will take place too slowly to be effective. The absorbent material acts to fill a virtual reservoir for the reaction. The absorbent material holds the water that feeds the reaction which generates the vapor.

Zeocal® 250 is known as a super absorbent polymer. Super absorbent polymers have an enormous capability to take on water (in the region of 600% of their specific mass). Super absorbent polymers are typically used in diapers, where the polymer pulls in and holds on to liquids. Similarly, in the present invention, the super absorbent polymer holds on to the water, whereas the exothermic agent wants to react with the water. This relationship helps to limit the rate of reaction to ensure that it lasts for the desired amount of time.

Furthermore, the amount of water that the super absorbent can take on is limited by the free volume left in the pouch 12 (and the amount that the pouch 12 will expand under pressure). By choosing a suitably stiff (non-expanding) material to form the pouch 12, it is possible to tightly regulate the amount of water that is available for the reaction to occur.

In one embodiment, the housing 14 is designed to hold about 10-12 ml of water. This water will provide enough “fuel” to sustain the reaction and give off fragrance for about 7-10 minutes. The temperature of the chemistry and surrounding water will reach the boiling point of water (212° F.) during the reaction. The steam generated from the boiling water carries the fragrance ingredient(s) into the environment, creating the desired environmental condition. The exterior of the housing 14 will reach around 0.190° F., but with the ridge design, the housing 14 can be handled relatively comfortably. This is important for purposes of handling the system 10 after activation of the exothermic reaction.

The pouch 12 may also be encased in an insulating material, such as a 3.0 mm thick cross-linked polyethylene foam. Insulating the pouch 12 allows for the heat generated by the reactant system 22 to be more effectively used to heat the water and create the fragrant vapor. The insulating material used in the present invention should have one or more of the following properties: be able to maintain its integrity at the elevated service temperature of 100° C.; be able to provide a temperature drop of at least 50° C. across its thickness; and be impermeable (for example, a closed cell foam) otherwise, the steam will simply pass through it. The pouch 12 may be placed in the insulating material at the time of manufacture, or the pouch 12 may be inserted into the insulating material and the housing 14 by the user just prior to exposing the vaporizing fragrance system to water.

The fragrance of the present embodiment fragrance system 10 may include menthol, camphor, eucalyptus, and lavender. These materials are known to provide a pleasant aroma when heated. The ratio of these materials can be varied by one of skill in the art and may also include natural fragrant oils (such as essential oils), plant or fruit extracts or distillates, discrete chemical compounds (such as various esters, lactones or ketones), and aqueous fragrant solutions. A variety of fragrant oils and other fragrant liquids are commercially available (both natural and synthetic), particularly those used in conventional potpourri products. These oils are typically formulated to provide a variety of pleasing aromas, such as: amber, apple, bayberry, bay rum, bitter almond, blueberry, blue magnolia, bubble gum, candy cane, cappuccino, carnation, coconut, chocolate, mocha, citrus punch, lilac, cucumber, eucalyptus, frankincense, gingerbread, heather, honeysuckle, jasmine, musk and pumpkin pie.

Other fragrant materials may also be incorporated, such as coco flowers, cinnamon, vanilla bean, hibiscus flowers, hollyhock flowers, karni, fern, rose leaves, tilia flowers, cloves, pine needles, cranberries, rhododendron leaves, rose hips, allspice, anise, casurina, and pomegranate.

Suitable aromatherapy fragrant materials include essential oils extracted from the following plants: ammi visnaga, angelica archangelica, basil linalol, ocimum basilicum, pimenta racemosa, laurus nobilis, bergamot, mint, mentha citrada, melaleuca cajeputi, daucus carota, cedrus atlantica, virginiana, chamomile, cistus ladanifer, salvia sclarea, citrus clementina, petitgrain, clove bud, coriandrum sativum, cypress, eucalyptus, fennel, frankincense, galanum, helichrysum italicum, lemon teatree, and vanilla.

As used herein, the term “fragrance” is not limited to just pleasing fragrances, but includes scents which function, for example, as deodorants and insect repellants (including scents which may be undetectable by the human olfactory system, such as scents used to attract or repel certain animals). One particular embodiment of the present invention comprises an air freshener for masking unpleasant odors (such as tobacco smoke) by emitting a pleasing fragrance. Another embodiment of the present invention comprises an aromatherapy device which emits aromatherapy fragrances (such as those emitted by various essential oils used for aromatherapy). Yet another embodiment comprises an insect repelling device which emits an insect repelling fragrance.

The embodiments described herein are for illustrative purposes and are not meant to exclude any derivations or alternative methods that are within the conceptual context of the invention. It is contemplated that various deviations can be made to these embodiments without deviating from the scope of the present invention. Accordingly, it is intended that the scope of the present invention be dictated by the appended claims rather than by the foregoing description of this embodiment.

1. A method of dispensing fragrant vapor into the air, comprising:
providing a fragrant material absorbed onto an absorbent material and a reactant system for generating heat when an aqueous solution is added to the reactant system, the fragrant material, absorbent material, and the reactant system in a container that includes one or more openings for allowing an aqueous solution to wet the fragrant material, the absorbent material, and the reactant system, the container providing for a temperature drop of at least about 30° C. along its thickness also including a number of ridges that allow for the dissipation of heat along an edge thereof, the temperature drop and the ridges allowing for comfortable handling of the container during the generation of heat by the reactant system; and
adding water to the container such that the reactant system generates heat and volatizes the fragrant material and thereby dispensing fragrant vapor into the air.

2. The method of claim 1 wherein wetting the container comprises immersing the container in water.

3. The method of claim 1 wherein wetting the container comprises hanging the container on the wall of a shower and allowing the water from the shower to fall onto the container.

4. The method of claim 1 wherein the reactant system comprises a mixture of magnesium and iron.

5. The method of claim 4 wherein the reactant system further comprises an electrolyte.

6. An effervescent delivery system comprising:
a pouch;
an effervescent material disposed within the pouch; and
a housing having a plurality of walls defining an interior space wherein the pouch is deposited, the housing also including a support structure adapted to position the effervescent material away from the walls.

7. The system of claim 6 wherein the effervescent material includes a reactant system and a fragrant mixture.

8. The system of claim 7 wherein the reactant system is a combination of supercorroding metallic materials.

9. The system of claim 8 wherein the supercorroding metallic materials are magnesium and iron.

10. The system of claim 6 wherein the reactant system further comprises a material of two or more metallic agents that can interact electronically to produce heat and an electrolyte.

11. The fragrance dispenser of claim 7 wherein the fragrant mixture includes a fragrant material absorbed into an absorbent.

12. The system of claim 6 wherein an interior surface of a wall includes a support structure adapted to position the effervescent material away from the interior surface of the walls.

13. The system of claim 6 wherein an exterior surface of at least one wall includes a ridge structure.

14. The system of claim 6, wherein at least one wall includes an aperture extending therethrough.

15. An fragrance delivery system for dispensing fragrance, comprising
a pouch;
an effervescent material comprised of a reactant system and a fragrant system, the effervescent material disposed within the pouch, and
a housing having a plurality of walls defining an interior space wherein the pouch is located and having a support structure adapted to position the effervescent material away from the walls, the housing also having at least one wall with a ridge structure located along an external surface.

16. The system of claim 15 wherein at least one wall has one or more openings that allow for water to enter the housing.

17. The system of claim 16 wherein the one or more openings designed to allow water to enter into the container are perforations.

18. The system of claim 15 wherein the container is made of a material that allows for a temperature drop of at least about 30° C. across its thickness.

19. The system of claim 15 wherein the reactant system is a combination of supercorroding metallic materials.

20. The system of claim 19 wherein the supercorroding metallic materials are magnesium and iron.

21. The system of claim 15 wherein the fragrant mixture further comprises an absorbent.

22. The system of claim 15 wherein the housing further comprises an insulator positioned inside the container and around the composition that includes the reactant system and the fragrant mixture.
